(19) United States
(12) Patent Application Publication
(10) Pub. No.: US 2006/0009445 A1
(43) Pub. Date: Jan. 12, 2006

(54) Tumor-inhibiting anellated azepinone derivatives

(75) Inventor: Bernhard Keppler, Gaweinstal (AT)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: Faustus Forschungs CIE. Translational Cancer Research GmbH

(21) Appl. No.: 11/165,153

(22) Filed: Jun. 23, 2005

(30) Foreign Application Priority Data
Dec. 23, 2002 (DE) 102 60 618.8

(51) Int. Cl. C07D 487/02; A61K 31/55

(52) U.S. Cl. 514/215; 540/577

(57) ABSTRACT

This invention relates to anellated azepinone derivatives, a method of their production, metal complexes of the anellated azepinone derivatives as well as their use in the treatment of tumor diseases.

### CROSS-REFERENCE TO RELATED APPLICATIONS

This application is a continuation of International Application No. PCT/EP2003/01483, filed Dec. 23, 2003, which was published in the German language on Jul. 15, 2004, under International Publication No. WO 2004/058766 A3 and the disclosure of which is incorporated herein by reference.

### BACKGROUND OF THE INVENTION

This invention relates to anellated azepinone derivatives, a method of their production, metal complexes of the anellated azepinone derivatives as well as their use in the treatment of tumor diseases.

The exact, step by step sequence of the individual steps of the cell cycle is an essential constituent of normal cellular proliferation. The various forms of cyclin dependent kinases (CDKs) are primarily responsible for the transition of the individual steps into one another. A deregulation of the activity of cyclin dependent kinases can be observed in many human tumors. These are based either on the overexpression of cyclins or also on the lack of appropriate natural inhibitors through genetic modifications. Consequently, CDKs represent an attractive class of targets for the chemotherapeutic treatment of tumor diseases. However, to date there are only few substances which are able to act as selective CDK inhibitors, wherein particularly flavopiridol, roscovitine and the purvalanols should be mentioned as important compounds or classes of compound.

Anellated azepinones represent a further important class of CDK inhibitors. The basic framework of this class of compounds facilitates a range of modifications for structural optimisation with a view to biological effectiveness. In particular, the class of the indolo[3,2-d]benzazepinones is a class of compounds, whose potential as CDK inhibitors is currently being intensively examined.

Consequently, in WO 99/65910 substituted azepinones were described as inhibitors in the cyclin dependent kinases. Also in WO 01/60374, the use of suitably substituted azepinone derivatives is disclosed for the manufacture of medicaments which contain inhibitors for GSK-3β, CDK1 or CDK5. Both in WO 99/65910 and in WO 01/60374 the substitution pattern on the two aryl rings of the tetracyclic basic structure of the corresponding azepinone derivatives was varied.

Replacing the lactam carbonyl group in the azepinone ring for a thioimidate or a hydroxyimidate grouping produced no improvement in the biological effectiveness (Schultz et al. J. Med. Chem. 1999, 42, 2909-2919).

### BRIEF SUMMARY OF THE INVENTION

The object of this invention is to make compounds which exhibit a high effectiveness available for the treatment of cancer diseases.

The object is solved by a compound of the general formula (I) or (II) 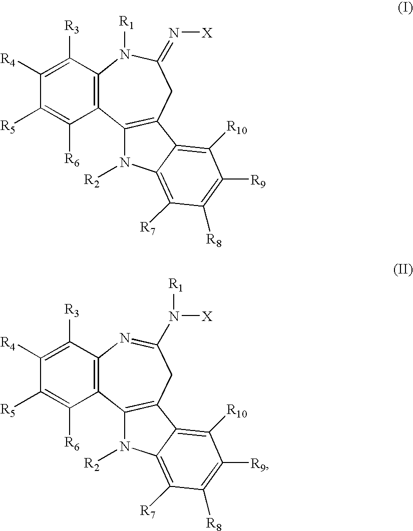
wherein
X is selected from the following groups (a), (b), (c) 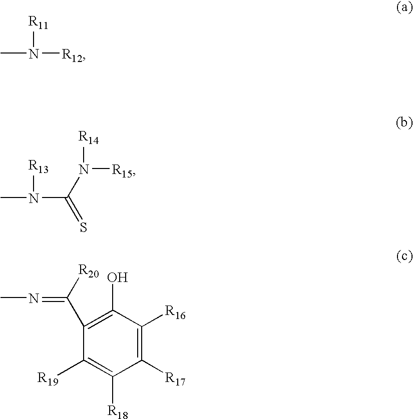
   and substituted or unsubstituted cycloalkyl, cycloalkenyl, aryl, substituted or unsubstituted, linear or branched alkyl, alkenyl and alkynyl,
wherein
R¹, R² and R¹¹-R¹⁵ are selected independently of one another from the group consisting of hydrogen, halogen, hydroxyl and substituted or unsubstituted cycloalkyl, cycloalkenyl, aryl, substituted or unsubstituted, linear or branched alkyl, alkenyl and alkynyl, and
R³-R¹⁰ and R¹⁶-R²⁰ are selected independently of one another from the group consisting of hydrogen, amino, nitro, cyano, formyl, carboxyl, SO₃H, SO₃M_{b}, wherein M_{b} is a physiologically compatible cation, hydroxy, halogen and substituted or unsubstituted cycloalkyl, cycloalkenyl, aryl, substituted or unsubstituted, linear or branched alkyl, alkenyl, alkynyl, alkoxy, alkylmercapto and dialkylamino, and
physiologically compatible addition salts thereof.

Furthermore, the object of this invention is solved by a complex of the general formula (III),
[Mₐⁱ⁺Yₓⁿ⁻L_{z}]^{[i-(nx+z)]+}[i−(nx+z)]/n Yⁿ⁻ (III)
wherein L is a group of the general formula (IV), 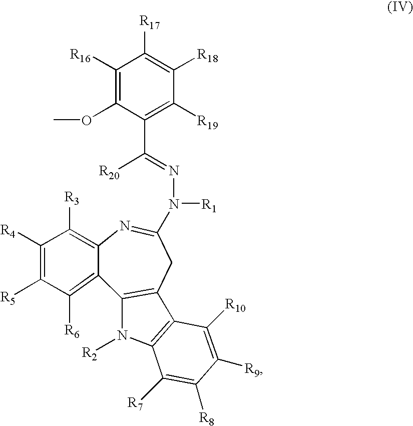
wherein
R¹ and R² are defined as above,
R³-R¹⁰ and R¹⁶-R²⁰ are defined as above, und
Mₐ is Ga, Fe, Ru or La,
Y is a physiologically compatible anion,
i is 2 or 3,
n is 1 or 2,
x is 0, 1, 2 or 3,
z is 1, 2 or 3,
nx+z≦i,
and physiologically compatible addition salts thereof.

Furthermore, this invention relates to a complex of the general formula (III′)
[MₐL₂]^{j+} j/p Y^{p-} (III′),
wherein
j is 0, 1 or 2,
p is 1 or 2, and
Mₐ, L and Y are defined as above.

Furthermore, the invention relates to a compound of the general formula (VI) 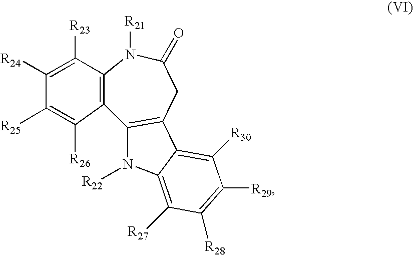
wherein
R²¹ and R²² are selected independently of one another from the group consisting of hydrogen, halogen, hydroxyl and substituted or unsubstituted cycloalkyl, cycloalkenyl, aryl, substituted or unsubstituted, linear or branched alkyl, alkenyl and alkynyl,
R²³ to R³⁰ are selected independently of one another from the group consisting of hydrogen, amino, nitro, cyano, formyl, carboxyl, hydroxy, halogen, SO₃H, SO₃ M_{b}, wherein M_{b} is a physiologically compatible cation, and substituted or unsubstituted cycloalkyl, cycloalkenyl, aryl, substituted or unsubstituted, linear or branched alkyl, alkenyl, alkynyl, alkoxy, alkylmercapto and dialkylamino, wherein at least one of the groups R²³ to R³⁰ is SO₃H or SO₃ M_{b}, wherein M_{b} is a physiologically compatible cation,
and physiologically compatible addition salts thereof.

Furthermore, this invention relates to a method for the manufacture of a complex of the general formula (III)
[Mₐⁱ⁺Yₓⁿ⁻L_{z}]^{[i-(nx+z)]+}[i−(nx+z)]/n Yⁿ⁻ (III)
wherein a compound L of the general formula (IV), 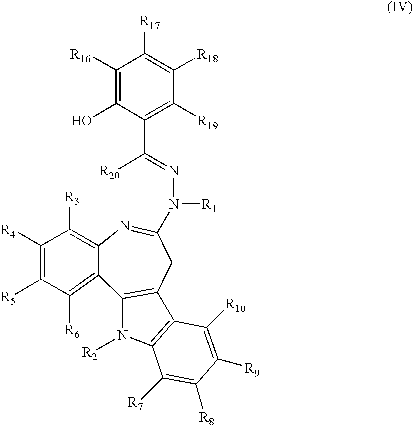
   wherein
R¹-R¹⁰ and R¹⁶-R²⁰ are defined as above,
is reacted with a compound of the general formula (V),
   MₐYₘ (V),
   wherein
Mₐ and Y are defined as above and m is 1, 2 or 3, and
i, n, x, and z are defined as above.

Furthermore, this invention relates to a method for the manufacture of a complex of the general formula (III′)
[MₐL₂]^{j+}j/p Y^{p-} (III′)
wherein a compound L of the general formula (IV), 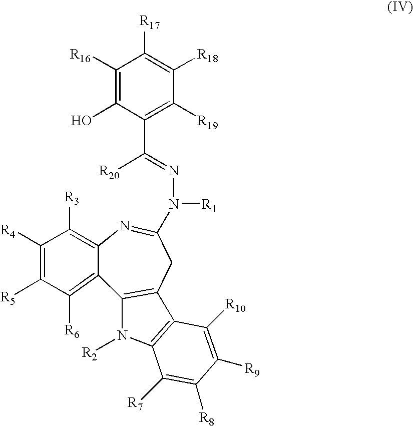
wherein
R¹ and R² are defined as above,
R³-R¹⁰ and R¹⁶-R²⁰ are defined as above,
is reacted with a compound of the general formula (V′),
   MₐY_{q} (V′),
   wherein
Mₐ is defined as above,
Y is a physiologically compatible anion,
q is 1, 2 or 3, and
j and p are defined as above.

Preferably, m or q is 3 when Y is a monovalent anion.

Furthermore, for the purpose of this invention alkyl preferably contains 1 to 10, more preferably 1 to 6 and especially 1 to 3 carbon atoms, alkenyl or alkynyl preferably contains 2 to 10, more preferably 2 to 6 and especially 2 to 3 carbon atoms, cycloalkyl or cycloalkenyl contains preferably 3 to 10, more preferably 3 to 8 and especially 3 to 6 carbon atoms and aryl preferably contains 6 to 14, more preferably 6 to 10 and especially 6 carbon atoms.

If the following groups of radicals R₁ and R₂; R₃ to R₆; R₇ to R₁₀; R₁₁, to R₁₅; R₁₆ to R₂₀; R₂₁ and R₂₂, R₂₃ to R₂₆; R₂₇ to R₃₀; and X are substituted in the general formulae (1), (II), (IV) or (VI), the substituents in these groups are, independently of one another, preferably halogen, hydroxyl, alkyl, alkoxy, alkoxycarbonyl, alkylmercapto, amino, dialkylamino, dialkylaminocarbonyl and/or nitrile, more preferably hydroxyl, amino and/or C₁-C₄-dialkylamino and especially hydroxyl and/or C₁-C₄-dialkylamino. Furthermore, preferably one to three substituents, in particular one substituent is present.

Also, substituted groups R³-R¹⁰, R¹⁶-R²⁰ and X in the general formulae (I), (II) or (IV) and R²³-R³⁰ in the general formula (VI) are preferably selected independently of one another from the group consisting of halogenalkyl, hydroxyalkyl, alkoxy, alkoxyalkylene, alkoxycarbonyl, alkoxycarbonylalkylene, alkylmercapto, alkylmercaptoalkylene, dialkylamino, dialkylaminoalkylene, dialkylaminocarbonyl, dialkylaminocarbonylalkylene und alkylnitrile.

Furthermore, preferably R¹, R² and R¹¹-R¹⁵ in the general formulae (I), (II) or (IV) and R²¹ and R²² in the general formula (VI) are selected independently of one another from the group consisting of hydrogen, C₁-C₆-alkyl, C₂-C₆-alkenyl, C₂-C₆-alkynyl, C₃-C₆-cycloalkyl, C₃-C₆-cycloalkenyl, C₆-C₁₄-aryl, halogen and hydroxyl, more preferably from hydrogen and C₁-C₆-alkyl. and especially hydrogen.

Furthermore, R³-R¹⁰, R¹⁶-R²⁰ in the general formulae (I), (II) or (IV) and R²³-R³⁰ in the general formula (VI) are preferably selected independently of one another from the group consisting of hydrogen, C₁-C₆-alkyl, C₂-C₆-alkenyl, C₂-C₆-alkynyl, C₃-C₆-cycloalkyl, C₃-C₆-cycloalkenyl, C₆-C₁₄-aryl, amino, nitro, cyano, formyl, carboxyl, SO₃H, SO₃ M_{b}, where M_{b} is a physiologically compatible cation, hydroxy, halogen, C₁-C₄-halogenalkyl, C₁-C₄-hydroxyalkyl, C₁-C₄-alkylnitrile, C₁-C₄-alkoxy, C₁-C₄-alkoxy-C₁-C₄-alkylene, C₁-C₄-alkylmercapto, C₁-C₄-alkylmercapto-C₁-C₄-alkylene, C₁-C₄-alkoxycarbonyl, C₁-C₄-alkoxycarbonyl-C₁-C₄-alkylene, di-C₁-C₄-alkyl-amino, di-C₁-C₄-alkylamino-C₁-C₄-alkylene, di-C₁-C₄-alkylaminocarbonyl and di-C₁-C₄-alkylaminocarbonyl-C₁-C₄-alkylene, more preferably from hydrogen, nitro, halogen and C₁-C₆-alkyl, and especially from hydrogen and halogen.

Furthermore, preferably R¹, R² and R¹¹-R¹⁵ in the general formulae (I), (II) or (IV) and R²¹ and R²² in the general formula (VI) are selected independently of one another from the group consisting of hydrogen, C₁-C₆-alkyl, C₂-C₆-alkenyl, C₂-C₆-alkynyl, C₃-C₆-cycloalkyl, C₃-C₆-cycloalkenyl, C₆-C₁₄-aryl, halogen and hydroxyl, more preferably from hydrogen and C₁-C₆-alkyl and especially hydrogen, and
R³-R¹⁰, R¹⁶-R¹⁰ in the general formulae (I), (I) or (IV) and R²³-R³⁰ in the general formula (VI) are selected independently of one another from the group consisting of hydrogen, C₁-C₆-alkyl, C₂-C₆-alkenyl, C₂-C₆-alkynyl, C₃-C₆-cycloalkyl, C₃-C₆-cycloalkenyl, C₆-C₁₄-aryl, amino, nitro, cyano, formyl, carboxyl, SO₃H, SO₃ M_{b}, where M_{b} is a physiologically compatible cation, hydroxy, halogen, C₁-C₄-halogenalkyl, C₁-C₄-hydroxyalkyl, C₁-C₄-alkylnitrile, C₁-C₄-alkoxy, C₁-C₄-alkoxy-C₁-C₄-alkylene, C₁-C₄-alkylmercapto, C₁-C₄-alkylmercapto-C₁-C₄-alkylene, C₁-C₄-alkoxycarbonyl, C₁-C₄-alkoxycarbonyl-C₁-C₄-alkylene, di-C₁-C₄-alkyl-amino, di-C₁-C₄-alkylamino-C₁-C₄-alkylene, di-C₁-C₄-alkylaminocarbonyl and di-C₁-C₄-alkylaminocarbonyl-C₁-C₄-alkylene, more preferably from hydrogen, nitro, halogen and C₁-C₆-alkyl, and especially from hydrogen and halogen. R¹-R⁶ and R¹¹-R²⁰ in the general formulae (I), (II) or (IV) are preferably hydrogen, and R⁷-R¹⁰ are selected independently of one another from the group consisting of hydrogen, C₁-C₆-alkyl, C₂-C₆-alkenyl, C₂-C₆-alkynyl, C₃-C₆-cycloalkyl, C₃-C₆-cycloalkenyl, C₆-C₁₄-aryl, amino, nitro, cyano, formyl, carboxyl, SO₃H, SQ3 M_{b}, where M_{b} is a physiologically compatible cation, hydroxy, halogen, C₁-C₄-halogenalkyl, C₁-C₄-hydroxyalkyl, C₁-C₄-alkylnitrile, C₁-C₄-alkoxy, C₁-C₄-alkoxy-C₁-C₄-alkylene, C₁-C₄-alkylmercapto, C₁-C₄-alkylmercapto-C₁-C₄-alkylene, C₁-C₄-alkoxycarbonyl, C₁-C₄-alkoxycarbonyl-C₁-C₄-alkylene, di-C₁-C₄-alkyl-amino, di-C₁-C₄-alkylamino-C₁-C₄-alkylene, di-C₁-C₄-alkylaminocarbonyl and di-C₁-C₄-alkylaminocarbonyl-C₁-C₄-alkylene, wherein at least one of the substituents R⁷-R¹⁰ is not equal to hydrogen.

R¹-R⁸ and R¹⁰-R²⁰ in the general formulae (I), (II) or (IV) are most especially hydrogen, and R⁹ is nitro, cyano, halogen or trifluoromethyl, and in particular R⁹ is a nitro group or a halogen, wherein bromine is especially preferred of the halogens.

Furthermore Mₐ in the general formulae (III), (III′), (V) or (V′) is preferably Ga.

In the general formula (VI) R²¹-R²⁸ and R³⁰ are especially preferred as hydrogen or C₁-C₆-alkyl, in particular hydrogen. R²⁹ is especially preferred as SO₃H or SO₃ M_{b}, where M_{b} is a physiologically compatible cation.

Moreover, R²¹-R²⁸ and R³⁰ in the general formula (VI) are preferably hydrogen and R²⁹ is preferably SO₃H or SO₃ M_{b}, where M_{b} is a physiologically compatible cation.

Furthermore, M_{b} is preferably sodium, potassium or ammonium.

Furthermore, X is preferably C₁-C₆-alkyl, C₂-C₆-alkenyl, C₂-C₆-alkynyl, C₃-C₆-cycloalkyl, C₃-C₆-cycloalkenyl or C₆-C₁₄-aryl. The substituents of X are preferably selected from hydroxyl, amino and C₁-C₄-dialkylamino.

X is especially preferred C₁-C₆-alkyl. The substituents of X are especially preferably selected from hydroxyl and C₁-C₄-dialkylamino.

X is especially C₁-C₆-alkyl and substituted by hydroxyl or C₁-C₄-dialkylamino.

The compounds, KP1428, KP1436, KP1437, KP1438, KP1472 and KP1473 shown in the examples, are particularly preferred. The compound KP1428 can contain CH₃OH from the synthesis. Furthermore, the compounds KP1437 and KP1438 can contain water and the compounds KP1472 and KP1473 can contain CH₃CH₂OH from the synthesis.

Organic or inorganic addition salts can be formed with the following anions: chloride, bromide, phosphate, carbonate, nitrate, perchlorate, sulphate, citrate, lactate, tartrate, maleate, fumarate, mandelate, benzoate, ascorbate, cinnamate, glycolate, methane sulphonate, formiate, malonate, naphthalene-2-sulphonate, salicylate and/or acetate.

H⁺, sodium, and/or potassium cations can be used as possible cations.

Preferably Y is selected from the group consisting of halogens, pseudo-halogens, nitrate, carboxylate, sulphate, carbonate, hydrogen phosphate, tartrate, malonate, oxalate and R″COO, where R″ is hydrogen, C₁-C₆-alkyl, C₂-C₆-alkenyl, C₂-C₆-alkynyl, C₃-C₆-cycloalkyl, C₃-C₆-cycloalkenyl, C₆-C₁₄-aryl, or a heterocycle.

Y is more preferably a halogen and most preferably chlorine.

The compounds or complexes according to the invention can contain water of crystallisation or solvent molecules such as methanol or ethanol.

Preferably i in the general formula (III) is 3. Furthermore, i is 2 or 3, if Mₐ is Ru or Fe, i.e. it is present in the oxidation stage II or III, and 3, if Mₐ is Ga, i.e. it is present in the oxidation stage III.

Preferably, n is 1 in the general formula (D). Furthermore, x is preferably 0 or 1 and in particular 0. In a further preferred embodiment z is 1 or 2 and especially 2.

Preferably in the general formula (III′) j is 0 or 1. Furthermore, j is 0 or 1, if Mₐ is Ru or Fe, i.e. it is present in the oxidation stage II or III, and 1, if Mₐ is Ga, i.e. it is present in the oxidation stage III.

The compound according to the invention can be used for the prophylaxis and/or treatment of cancer diseases.

### DETAILED DESCRIPTION OF THE INVENTION

In the following the medicament containing a compound according to the invention is described in more detail.

The medicament according to the invention is primarily administered intravenously, but also intramuscularly, intraperitoneally, subcutaneously or perorally. External application is also possible. Preferably, it is administered by intravenous injection or by intravenous infusion.

The medicament is manufactured according to known methods, whereby the compound according to the invention is used as such or optionally in combination with suitable pharmaceutical carrier substances. If the medicament according to the invention contains pharmaceutical carrier substances as well as the active substance, the content of active substance in this mixture is 0.1 to 99.5, preferably 0.5 to 95% by weight of the total mixture.

The medicament according to the invention can be applied in any suitable formulation with the prerequisite that the establishment and maintenance of a sufficient level of active substance is ensured. This can, for example, be achieved by the oral or parenteral administration in suitable doses. Advantageously, the pharmaceutical preparation of the active substance is provided in the form of standard doses which are matched to the desired administration. A standard dose can, for example, be a tablet, a coated tablet, capsule, suppository or a measured volume of a powder, granulate, solution, emulsion or suspension.

A “standard dose” for the purposes of this invention is taken to mean a physically determined unit which contains an individual quantity of the active constituent in combination with a pharmaceutical carrier substance and its content of active substance corresponds to a fraction or multiple of a therapeutic single dose. A single dose preferably contains the quantity of active substance which is administered during an application and which normally corresponds to a whole, half, third or quarter of the daily dose. If only a fraction, such as half or quarter of the standard dose is needed for a single therapeutically administered dose, then the standard dose is advantageously divisible, e.g. in the form of a tablet with a dividing groove.

The medicaments according to the invention can, if the active substance is present in standard doses and is intended for application, e.g. on persons, contain about 0.1 to 500 mg, preferably 10 to 200 mg and particularly 50 to 150 mg of active substance.

Generally in human medicine, the active substance(s) is/are administered in a daily dose of 0.1 to 5, preferably 1 to 3 mg/kg of body weight, optionally in the form of a number, preferably 1 to 3, of single intakes for achieving the desired results. A single intake contains the active substance(s) in quantities of 0.1 to 5, preferably 1 to 3 mg/kg of body weight. With oral treatment similar dosages can be applied.

The therapeutic administration of the medicament according to the invention can occur 1 to 4 times daily at specified or varying time points, e.g. in each case before meals and/or in the evening. However, it may be necessary to deviate from the quoted dosages depending on the type, body weight and age of the individual to be treated, the type and severity of the disease, the type of preparation and the application of the medicament as well as the time period or interval within which the administration occurs. Consequently, in some cases it may be sufficient to use less than the amount of active substance mentioned above, whereas in other cases the above listed quantity of active substance must be exceeded. It may also be practicable to administer the medicaments only once or at intervals of a number of days.

The specification of the necessary optimum dosage and type of application of the active substances can be made by any person skilled in the art based on his/her specialist knowledge.

The medicaments according to the invention normally comprise the compounds according to the invention and non-toxic, pharmaceutically compatible medicament carriers, which as additive or dilution agents, are employed, for example, in solid, semi-solid or liquid form or as a means of enclosure, for example in the form of a capsule, a tablet coating, a bag or another container for the therapeutically active constituent. A carrier substance may, for example, act as an intermediary for the ingestion of the medicament by the body, as an auxiliary formulation agent, sweetener, taste modifier, colorant or as a preservative.

For oral application, for example, tablets, coated tablets, hard and soft capsules, for example of gelatine, dispersible powder, granulate, aqueous and oily suspensions, emulsions, solutions and syrups can be employed.

Tablets can contain inert filling agents, e.g. calcium carbonate, calcium phosphate, sodium phosphate or lactose; granulation and distribution agents, e.g. maize starch or alginates; binding agents, e.g. starches, gelatine or arabine; and lubricating agents, e.g. aluminium or magnesium stearate, talc or silicone oil. They can additionally be provided with a coating which is produced such that it causes delayed release and resorption of the medicament in the gastro-intestinal tract, so that, for example, improved compatibility, assimilation or retardation is achieved. Gelatine capsules may contain the pharmaceutical substance mixed with a solid, e.g. calcium carbonate or kaolin or an oily dilution agent, e.g. olive, peanut or paraffin oil.

Aqueous suspensions can contain suspension agents, e.g. sodium carboxymethyl cellulose, methyl cellulose, hydroxypropyl cellulose, sodium alginate, polyvinyl pyrrolidon, traganth rubber or arabine; dispersant or wetting agents, e.g. polyoxyethylene stearate, heptadeca-ethylene-oxycatanol, polyoxyethylene sorbitol-monooleate, or lecithin; preservatives, e.g. methyl- or propylhydroxy-benzoate; taste modifiers; sweeteners, e.g. saccharose, lactose, sodium cyclamate, dextrose, invert sugar syrup.

Oily suspensions may contain, for example, peanut, olive, sesame, coconut or paraffin oil and thickening medicaments, such as bees wax, high melting point wax or cetyl alcohol; also sweeteners, taste modifiers and antioxidants.

Powder and granulates dispersible in water may contain the compound according to the invention in a mixture with dispersing, wetting and suspension agents, e.g. those mentioned above as well as with sweeteners, taste modifiers and colorants.

Emulsions can, for example, contain olive, peanut or paraffin oil as well as emulsifying agents such as arabine, traganth rubber, phosphatides, sorbitan monooleate, polyoxyethylene sorbitan monooleate and sweeteners and taste modifiers.

Aqueous solutions can contain preservatives, e.g. methyl- or propylhydroxybenzoates; thickening agents; taste modifiers; sweeteners, e.g. saccharose, lactose, sodium cyclamate, dextrose, invert sugar syrup as well as taste modifiers and colorants.

For the parenteral application of pharmaceutical substances sterile injectable aqueous solutions, isotonic salt solutions or other solutions can be used.

The following examples explain the invention.
Reaction of Anthranilic Acid Ethyl Ester with Succinic Acid Ethyl Ester (1) (JACS, 1958, 80, 2172):

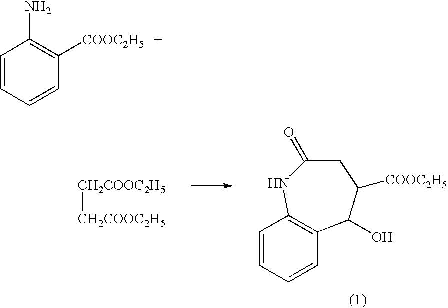

A mixture consisting of 14.7 g of succinic acid ethyl ester, 10.3 g of anthranilic acid ethyl ester, and 2.14 g of sodium hydride were heated in 140 ml of dry toluol for 3 hours with stirring under reflux. The reaction mixture was allowed to stand overnight and then 75 ml of 10% hydrochloric acid was slowly added. The precipitate formed was filtered off and recrystallised three times out of ethanol. Yield: 4.5 g (29.5%), Mp.: 210-213° C.; IR: 1673 cm−1 (conjugated ester), 1648 cm−1 (amide), 1623 cm−1 (C═C); λmax(ethanol) 228-229 nm (ε=29260), 239 nm (ε=19580) sh, 293-296 nm (ε=12200). Anal. Calc. for C12H13NO4 (235.24): C, 63.15; H, 5.30; N, 5.67. Fnd.: C, 63.31; H, 5.45; N, 5.78.

### 2,3,4,5-tetrahydro-1H-1-benzazepine-2,5-dion (Arch. Pharm. 1991, 324, 579):

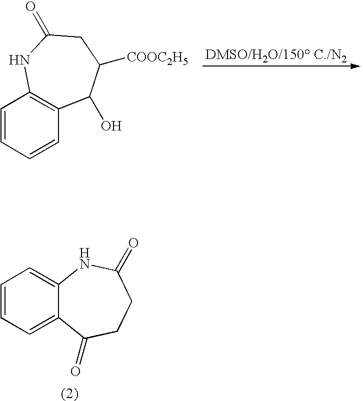

494 mg (2 mmol) of 1 were heated to 150° C. with 0.07 ml (3.9 mmol) of water in 10 ml of DMSO with stirring under N₂. After 1 h it was poured into 50 ml of water and extracted 10 times, each time with 10 ml of CH₂Cl₂. The combined organic phases were washed with water, dried over Na₂SO₄ and evaporated down in a vacuum. Recrystallisation of the residue from ethanol gave colourless crystals. Yield 85%, Mp.: 187-188° C. (EtOH). C₁₀H₉NO₂ (175.2); IR: 3220 (NH), 1660 cm⁻¹ (C═O); ¹H NMR (DMSO-d₆) δ (ppm): 10.05 (bs; 1H, NH), 7.81 (dd; 1H, J=1.5/8 Hz, Hₐᵣₒₘₐₜ), 7.53 (ddd; 1H, J=1.5/7/8 Hz, Hₐᵣₒₘₐₜ), 7.19-7.13 (m; 2Hₐᵣₒₘₐₜ), 2.63-2.93 (m, AA′BB′; 4H, CH₂CH₂). EI MS: m/z (%)=175 (93%, M⁺).

### 9-bromo-7,12-dihydro-indolo[3,2-d][1]benzazepine-6(5H)-on (3) (Arch. Pharm. 1992, 325, 297)

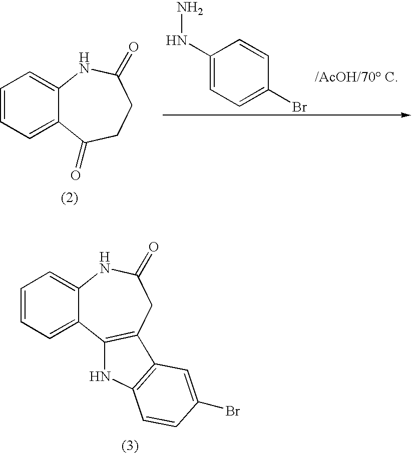

Bromophenylhydrazine (7 mmol) was added to a suspension of 2 (1.05 g, 6 mmol) in glacial acetic acid (10 ml) and then stirred for 1 h at 70° C. After cooling down it was stirred with 0.5 ml conc. H₂SO₄ for 1 h at 70° C. It was allowed to cool, poured into 50 ml of 10 percent sodium acetate solution and the precipitate was drawn off. Yellow crystals. Yield: 58%. Mp.: >330° C. (1,4-dioxane). C₁₆H₁₁BrN₂O (327.2). IR: 3220 (NH); 1640 cm⁻¹ (C═O). ¹H NMR: δ (ppm): 11.75 (s; 1H, NH), 10.05 (s; 1H, NH), 7.89 (d; 1H, J=1.5 Hz, C-8-H), 7.74 (bd; 1H, J=7.5 Hz, Ar—H), 7.41-7.34 (m; 2H, Ar—H), 7.30-7.21 (m; 3H, Ar—H), 3.50 (s; 2H, CH₂).

### 9-bromo-7,12-dihydroindolo[3,2-d][1]benzazepine-6-(5H)-thion (4) J. Med. Chem. 1999, 42, 2909)

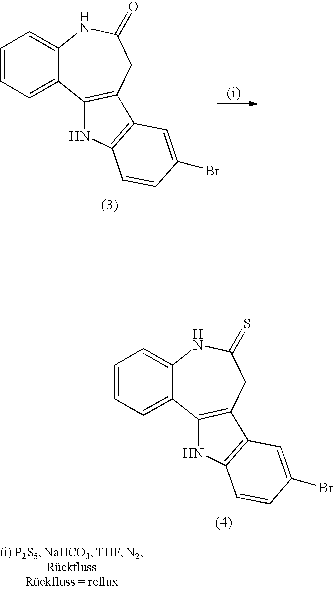

A solution of 3 (327 mg, 1 mmol) was stirred in THF (30 ml) at 50° C. under a nitrogen protective gas atmosphere. Phosphorus pentasulphide (250 mg, 1.12 mmol) and sodium hydrogencarbonate (370 mg, 4.4 mmol) were added consecutively. After three hours of heating under reflux in a nitrogen protective gas atmosphere, the reaction mixture was allowed to cool to room temperature and the reaction mixture was then placed on ice (50 g). Stirring took place until the ice had completely melted. Then the precipitate formed was drawn off, washed with water and recrystallised out of ethanol/toluol. Yield: 67% of slightly yellow crystals: Mp.: >330° C.; IR: 3430, 3140 cm⁻¹ (NH); ¹H NMR (400 MHz) 3.91 (s, 2H, CH₂), 7.30 (dd, 1H, 1.5/8.6 Hz), 7.39-7.45 (m, 4H), 7.79 (d, 1H, 7.1 Hz), 7.86 (d, 1H, 1.5 Hz), 11.92 (s, 1H, NH), 12.07 (s, 1H, NH).

### 9-bromo-6-(methylthio)-7,12-dihydroindolo[3,2-d][l]-benzazepine (5) J. Med. Chem. 1999, 42, 2909)

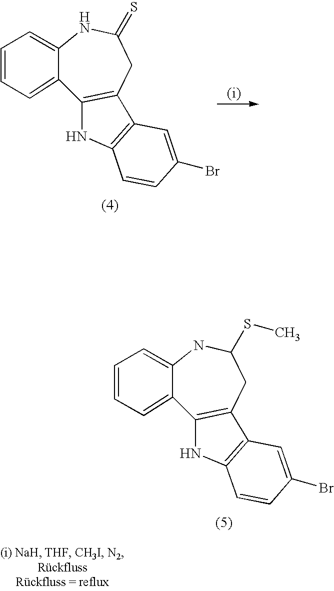

Sodium hydride (24 mg, 1 mmol, 60% suspension in oil) was added to a solution of 4 (343 mg, 1 mmol) in THF (20 ml). After heating for 1 hour under reflux with stirring in a nitrogen protective gas atmosphere, the mixture was cooled to room temperature and a solution of iodomethane (170 mg, 1.2 mmol) in THF (2 ml) added. Heating then took place for a further 2 hours under reflux with cooling to room temperature and then the reaction mixture was placed on ice (150 ml). After 15 minutes of stirring, the precipitate was drawn off, washed with water and crystallised out of ethanol. Yield: 44% of colourless crystals; Mp.: 199° C.; IR: 3420 (NH), 1615 cm⁻¹ (C═N); ¹H NMR (400 MHz) 2.35 (s, 3H, CH₃), 3.51 (s, 2H, CH₂), 7.26-7.32 (m, 2H), 7.36-7.43 (m, 3H), 7.80-7.82 (m, 1H), 7.97 (d, 1H, 1.5 Hz), 11.82 (s, 1H, NH).

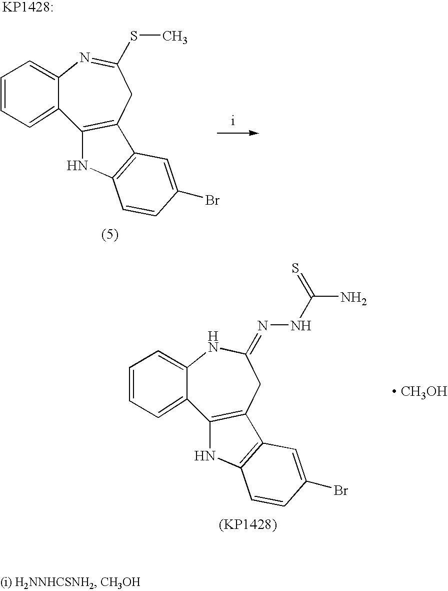

Thiosemicarbazide (220 mg) in methanol (55 ml) was added to 5 (714 mg, 2 mmol). The solution was filtered and stored at room temperature. After four days the precipitate formed was filtered off, washed with methanol and dried in air. Yield: 490 mg. Calc. for C₁₈H₁₈N₅BrOS, %: C, 50.01; H, 4.20; N, 16.20. Fnd., %: C, 50.12, H, 4.55; N, 15.63. MS (ESI): m/z=400 [M+].

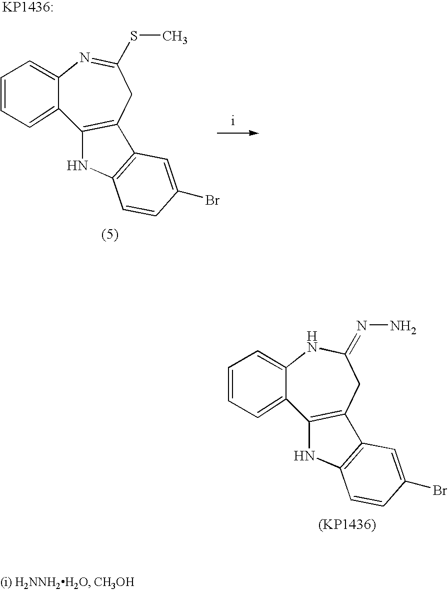

Hydrazine hydrate (0.2 ml) was added to 5 (714 mg, 2 mmol) in dry ethanol (50 ml). The solution was filtered and then stored at room temperature. After two days the precipitate formed was filtered off, washed with ethanol and dried in air. Yield: 236 mg. Calc. for C₁₆H₁₃N₄Br, %: C, 56.32; H, 3.84; N, 16.42. Fnd., %: C, 56.19, H, 3.80; N, 16.22. MS (ESI): m/z=341 [M⁺].

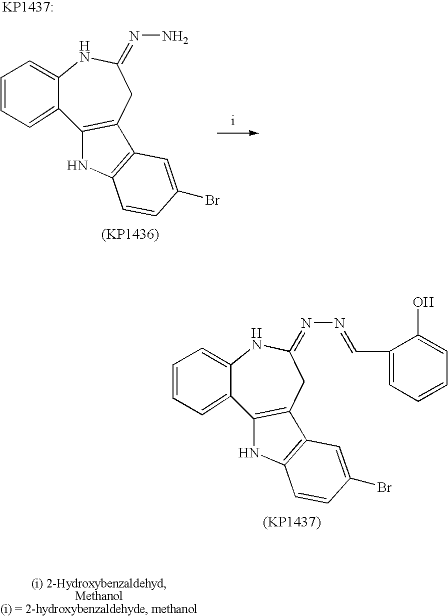

2-hydroxybenzaldehyde was added to KP1436 in boiling methanol (30 ml) and the solution was heated until all the starting material was dissolved. On the next day the precipitate formed was filtered off, washed with methanol and dried in air. Yield: 150 mg. Calc. for C₂₃H₁₉N₄O₂Br, %: C, 59.62; H, 4.13; N, 12.09. Fnd.: %: C, 60.23, H, 4.17; N, 12.12. MS (ESI): m/z=445 [M⁺].

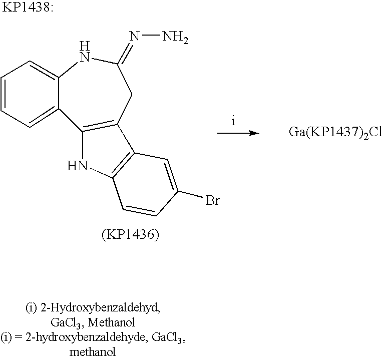

KP1436 (170 mg, 4.9 mmol) was heated in methanol (30 ml) to boiling and 2-hydroxybenzaldehyde (60 mg, 4.9 mmol) in methanol (1 ml) was added. The reaction mixture was heated under reflux for 15 min. Once the educt had completely dissolved, KP1437 started to crystallise out. At this point a solution of GaCl₃ (2.45 mmol) in ethanol (0.3 ml) was added. The solution thus obtained was heated for 20 min under reflux and then allowed to stand at room temperature. On the next day the precipitate formed was filtered off, washed with cold methanol and dried in a vacuum. Yield: 90 mg. The yield can be improved to 140 mg when the reaction is carried out in the presence of 0.05 g of triethylamine. Calc. for C₄₆H₃₂N₈GaBr₂ClO₂.2.5H₂O, %: C, 53.19; H, 3.59; N, 10.79. Fnd., %: C, 52.89, H, 3.60; N, 10.63. MS (ESI): m/z=957 [M⁺-Cl].

### 9-bromo-6-N-(2-N′,N′-dimethylaminoethylamino)-7,12-dihydroindolo-[3,2-d][l]benzazepine (KP1472)

### (9-bromo-6-N-(2-N′,N′-dimethylaminoethylamino)-7,12-dihydro-benzo[2,3]azepino[4,5-b]indol)

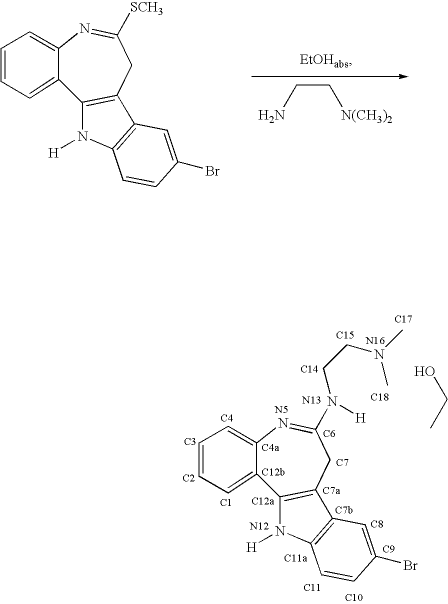 Formulation

1.50 g 9-bromo-6(methylthio)-7,12-dihydroindolo[3,2-d][l]benzazepine (M=357,2 g mol⁻¹=>4.2 mmol)

500 μl N,N-dimethylethylene diamine (95%; d=0.803 g cm⁻³; M=88.15 g mol⁻¹=>4.3 mmol)

50 ml Ethanol (abs.)

9-bromo-6(methylthio)-7,12-dihydroindolo[3,2-d][l]benzazepine (1.50 g) was placed into a 100 ml round flask with gas tap, dissolved in absolute ethanol (50 ml) with the aid of ultrasound and N,N-dimethylethylene diamine (0,5 ml) was added using a piston pipette (Eppendorf). Then the flask was closed off under argon with a rubber septum and placed in the drying cabinet (57-61° C.). After 14 days it was taken out of the drying cabinet and the reaction mixture filtered through a glass sintered strainer. Then, at 57-60° C. oil-bath temperature under suction of air, constriction down to about ¼ slowly followed through a delivery tube with wadding plugs, wherein the product precipitated as fine crystals. Then cooling took place in the freezing cabinet (−20° C.) before filtering in a strainer, washing three times with in each case 10 ml of cooled (−20° C.) ethanol (96%) and drying under suction.

Yield: 1.1 g of colourless crystalline product (58.9% of the theory); product contains an equivalent of ethanol).

Ethanol was removed before the NMR measurement in a vacuum.

δ_{H}(4000.13 MHz; d₆-DMSO):

11.61 [1H(N12); s]; 7.8 [1H(C8); s]; 7.68 [1H(C1); d; ³J(H_{C2})=7.53 Hz]; 7.36 [1H(C11); d; ³J(_{C10})=8.53 Hz]; 7.25 [1H(C3); dd; ³J(H_{C2})=8.53 Hz; ³J(H_{C4})=8.03 Hz]; 7.23 [1H(C10); d; ³J(H_{C11})=8.53 Hz];

7.14 [1H(N13); t; ³J(H_{C14})=5.02 Hz]; 7.13 [1H(C4); d; ³J(H₃)=8.03 Hz]; 7.03 [1H(C2); dd; ³J(H_{C1})=7.53 Hz; ³J(H_{C3})=8.53 Hz]; 3.32 [2H(C7); s]; 3.28 [2H(C14); dt; ³J(H_{N13})=5.02 Hz; ³J(H_{C15})=6.53 Hz]; 2.15 [6H(C17+C18); s];

δ_{C}(100.63 MHz; d₆-DMSO):

155.52(C6); 147.25(C4a); 136.78(C11a); 136.24(C12a); 129.09(C7b); 128.36(C4); 128.23(C3); 127.42(C1); 124.49(C10); 122.59(C12b); 121.07(C2); 121.00(C8); 113.96(C11); 112.01(C9); 109.31(C7a); 58.61(C15); 46.12(C17+C18); 39.90(C14); 28.66(C7);

Elementary analysis (C₂₀H₂₁N₄Br.C₂H₆O):

| | w-% C | w-% H | w-% N |
|---|---|---|---|
| Theoretical | 59.60 | 6.14 | 12.64 |
| Found | 59.68 | 5.85 | 12.93 |

### 9-bromo-6-(2-hydroxyethylamino)-7,12-dihydroindolo[3,2-d][l]benzazepine (KP1473)

### (9-bromo-6-(2-hydroxyethylamino)-7,12-dihydro-benzo[2,3]azepino[4,5-b]indol)

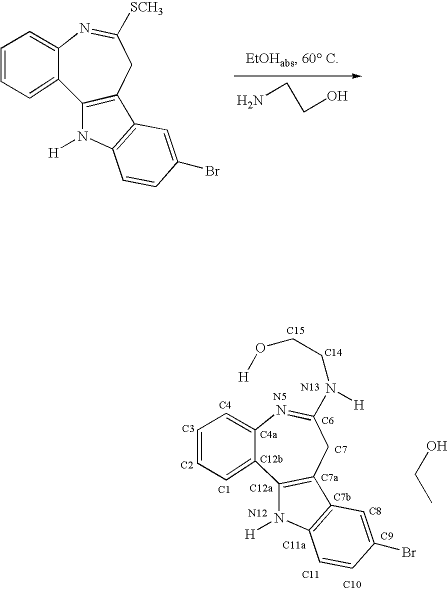 Formulation

1.50 g 9-bromo-6(methylthio)-7,12-dihydroindolo[3,2-d][l]benzazepine (M=357.2 g mol⁻¹=>4.2 mmol)

270 μl 2-aminoethanol (99%; d=1.015 g cm⁻³; M=61.08 g mol⁻¹=>4.4 mmol) 50 ml Ethanol (abs.)

9-bromo-6(methylthio)-7,12-dihydroindolo[3,2-d][l]benzazepine (1.50 g) was dissolved in a 100 ml round flask with gas tap in absolute ethanol (50 ml) with the aid of ultrasound. Ethanolamine was added using a piston pipette (Eppendorf). Then the flask was closed off under argon with a rubber septum and placed in the drying cabinet (57-60° C.). After 14 days it was taken out of the drying cabinet and filtered through a glass sintered strainer. Then, constriction took place slowly at 57-60° C. oil-bath temperature under suction of air through a deliver tube with wadding plugs until almost dry. The product thus obtained (small intergrown crystals) was suspended in 5 ml of cold ethanol, immediately filtered through a glass sintered strainer and washed again briefly with 5 ml of ethanol (96%). Then further drying took place in a vacuum.

Yield: 0.8 g (47.4% of theory); the product contains an equivalent of ethanol.

δ_{H}(400.13 MHz; d₆-DMSO):

11.62 [1H(N12); s]; 7.82 [1H(C8); s]; 7.68 [1H(C1); d; ³J(H_{C2})=8.03 Hz]; 7.36 [1H(C11); d; ³J(_{C10})=8.53 Hz]; 7.35 [1H(N13); t; ³J(H_{C14})=5.02 Hz]; 7.25 [1H(C3); dd; ³J(H_{C2})=8.53 Hz; ³J(H_{C4})=8.03 Hz];

7.23 [1H(C10); d; ³J(H_{C11})=8.53 Hz]; 7.11 [1H(C4); d; ³J(H_{C3})=8.03 Hz]; 7.04[1H(C2); dd; ³J(H_{C1})=8.03 Hz; ³J(H_{C3})=8.53 Hz]; 4.94 [1H(O); s]; 4.36 [1H(EtOH—OH); t; ³J(H_{EtOH—CH2}) 5.02 Hz]; 3.52 [2H(C15); t; ³J(H_{C14})=5.52 Hz]; 3.45 [2H(EtOH—CH2); dq; ³J(H_{EtoH—OH})=5.02 Hz; ³J(H_{EtOH—CH3})=7.03 Hz]; 3.32 [2H(C7); s]; 3.26 [2H(C14); dt; ³J(H_{C15})=5.52 Hz; ³J(H_{N13})=5.02Hz]; 1.06[3H(EtOH—CH3); t; ³J(_{HEtOH—CH2})=7.03 Hz];

δ_{c}(100.63 MHz; d₆-DMSO):

156.06(C6); 147.02(C4a); 136.81(C11a); 136.21(C12a); 129.06(C7b); 128.28(C4); 128.23(C3); 127.47(C1); 124.54(C10); 122.66(C12b); 121.21(C2); 120.99(C8); 114.00(C11); 112.05(C9); 109.31(C7a); 60.82(C15); 56.91(EtOH—CH2); 44.93(C14); 28.66(C7); 19.43(EtOH—CH3);

Elementary analysis (C₁₈H₁₆N₃OBr.C₂H₆O):

| | w-% C | w-% H | w-% N |
|---|---|---|---|
| Theoretical | 57.70 | 5.33 | 10.09 |
| Found | 57.71 | 5.32 | 10.14 |

### Sodium-6(5H)-oxo-7,12-dihydroindolo[3,2-d][1]benzazepine-9-sulphonate (KP1474)

### (Sodium-6-oxo-5,12-dihydro-7H-benzo[2,3]azepino[4,5-b]indol-9-sulphonate)

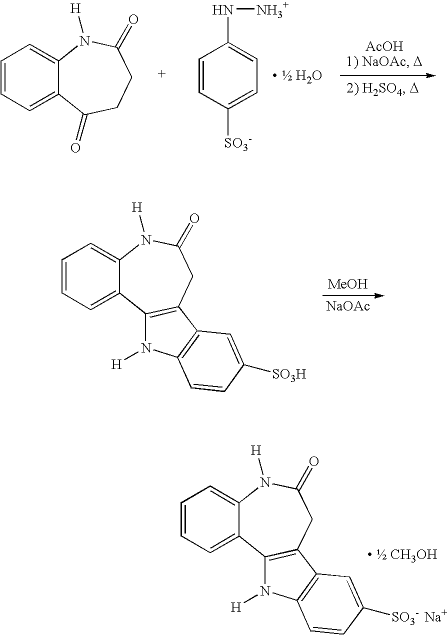 Formulation

0.50 g Dihydro-1H-benz[b]azepine-2,5-dion (M=175.19 g mol⁻¹=>2.85 mmol)

0.57 g 4-hydrazinobenzenesulphonic acid hemihydrate (98%; M=197.22 g mol⁻¹-=>2.8528 mmol)

0.24 g Sodium acetate (98.5%, M=82.034 g mol⁻¹=>2.88 mmol)

4.5 ml Acetic acid (99.8%; d=1.049 g cm⁻³)

250 μl H₂SO₄ (95-97%; d=1.84 g cm⁻³; M=98.079 g mol⁻¹=>4.50 mmol)

0.79 g Sodium acetate (98.5%, M=82.03 g mold =>9.48 mmol)

325 ml Methanol (for analysis) 7.6 ml H₂O

Dihydro-1H-benz[b]azepine-2,5-dion and sodium acetate were charged into a 10 ml round flask and suspended in 3 ml of glacial acetic acid with the aid of ultrasound. 4-hydrazinobenzene sulphonic acid hemihydrate in solid form was added while stirring. The mixture was then subject to reflux under an argon protective gas atmosphere for 75 min at 133° C. oil-bath temperature. The educts almost completely dissolved. Then the still hot reaction mixture was filtered through a glass sintered strainer (P4), wherein rinsing took place with 1.5 ml of acetic acid. After cooling to room temperature 250 μl of sulphuric acid was added using a piston pipette while stirring. Already after a very brief ultrasound treatment, a fine, bright precipitate started to form. Now boiling occurred in a hot oil bath under reflux for a further 75 min. under argon. After cooling to room temperature, filtering took place in a glass sintered strainer, then washing with 3 ml of glacial acetic acid, then three times, each time with 5 ml of THF and finally three times, each time with 5 ml of diethyl ether.

The product (about 1 g), which was dried under suction for some time and which according to experience contained some impurities which were difficult to identify, was dissolved in 300 ml of methanol with 7 ml of H₂O. For this, a solution of three equivalents of sodium acetate was poured into 25 ml of methanol with 0.6 ml of H₂O. The ensuing precipitate which formed was filtered off and discarded. The filtrate was centrifuged until dry and the residue suspended in 30 ml of dry methanol under ultrasound. After 10 min. of refluxing, the white product in the mixture which had cooled to room temperature was filtered off in a glass sintered strainer and washed three times with 5 ml of cold methanol each time and then three times with 5 ml of diethyl ether each time. Then drying took place in a vacuum.

Yield: 0.62 g of white powder

δ_{H}(400.13 MHz; d₆-DMSO):

11.69[1H(N12); s]; 10.12[1H(N5); s]; 7.91[1H(C8); s];

7.76[1H(C1); d; ³J(H_{C2})=7.5 Hz]; 7.51[1H(C10); d; ³J(H_{C11})=8.5 Hz];

7.39[1H(C11); d; ³J(_{C10})=8.5 Hz]; 7.37[1H(C3); dd; 3J(H_{C2})=7.5 Hz; ³J(H_{C4})=7.5 Hz];

7.28[1H(C2); dd; ³J(H_{C1})=7.5 Hz; ³J(H_{C3})=7.5 Hz;]; 7.27[1H(C4); d; ³J(H_{C3})=7.5 Hz];

4.12[0.5H(O_{MeOH}); s]; 3.49[2H(C7); s]; 3.18[1.5H(C_{MeOH}); s];

δ_{C}(100.63 MHz; d₆-DMSO):

172.27(C6); 140.67(C9); 138.15(C11a); 136.33(C12b); 134.12(C12a); 128.96(C3); 127.75(C1); 126.10(C7b); 124.57(C2); 123.62(C4a); 123.17(C4); 121.39(C10); 116.07(C8); 111.31(C11); 108.97(C7a); 49.35(C_{MeOH}); 32.56(C7);

δ_{N}(40.55 MHz; referred to NH₄Cl; d₆-DMSO):

108(N12); 117(N5);

Tumor-Inhibiting Effect:

The tumor-inhibiting effect was tested in vitro in the XTT assay with an example of various cell lines. The incubation period was 48 hours (IC₅₀ values in μM).

IC₅₀ values of azepinone derivatives

| Line | KP 1428 | KP1437 | KP1438 |
|---|---|---|---|
| CCRF-CEM | 5.47 | 1.66 | 0.54 |
| K-562 | 48.4 | 1.56 | 0.61 |
| MOLT-4 | 2.63 | 2.9 | 0.35 |
| COLO 205 | 13.3 | 5.25 | 1.85 |
| HCT-15 | 24.1 | 0.74 | 0.15 |
| HCT-116 | 26.4 | 2.56 | 1.32 |
| HT-29 | 9.46 | 6.61 | 1.64 |
| SW-620 | 22.8 | 3.83 | 2.57 |
| MCF-7 | 20.7 | 10.4 | 6.32 |
| OVCAR 3 | 2.59 | 6.82 | 4.08 |
| NCI-H460 | 14.4 | 9.51 | 5.35 |
| NCI-H226 | 30.1 | 7.19 | 2.04 |
| SK-MEL-5 | 19.4 | 10 | 0.64 |
| SK-MEL-28 | 18.5 | 7.98 | 0.44 |
| Hep 3B | | | 0.935 |
| Hep G2 | | | 0.385 |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. Compound of the general formula (I) or (II) 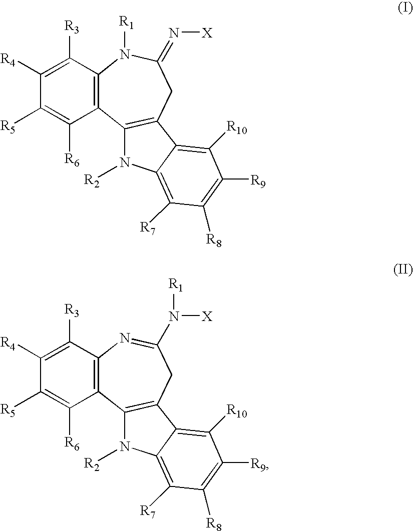
wherein
X is selected from the following groups (a), (b), (c) 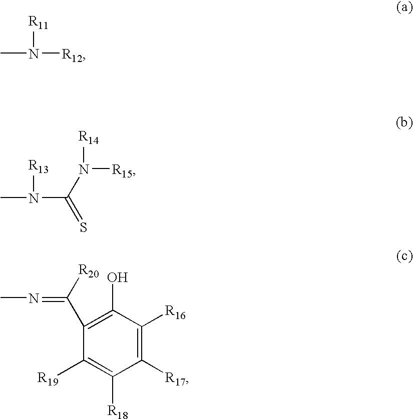
and substituted or unsubstituted cycloalkyl, cycloalkenyl, aryl, substituted or unsubstituted, linear or branched alkyl, alkenyl and alkynyl,
wherein
R¹, R² and R¹¹-R¹⁵ are selected independently of one another from the group consisting of hydrogen, halogen, hydroxyl and substituted or unsubstituted cycloalkyl, cycloalkenyl, aryl, substituted or unsubstituted, linear or branched alkyl, alkenyl and alkynyl, and
R³-R¹⁰ and R⁶-R²⁰ are selected independently of one another from the group consisting of hydrogen, amino, nitro, cyano, formyl, carboxyl, SO₃H, SO₃ M_{b}, wherein M_{b} is a physiologically compatible cation, hydroxy, halogen and substituted or unsubstituted cycloalkyl, cycloalkenyl, aryl, substituted or unsubstituted, linear or branched alkyl, alkenyl, alkynyl, alkoxy, alkylmercapto and dialkylamino, and
physiologically compatible addition salts thereof.

2. Compound according to claim 1, wherein X is selected from the following groups (a), (b), (c)

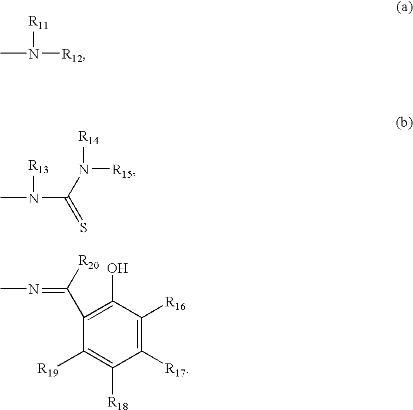

3. Complex of the general formula (E),
[Mₐⁱ⁺Yₓⁿ⁻L_{z}][i−(nx+z)]+[i−(nx+z)]/n Yⁿ⁻ (III)
wherein L is a group of the general formula (IV), 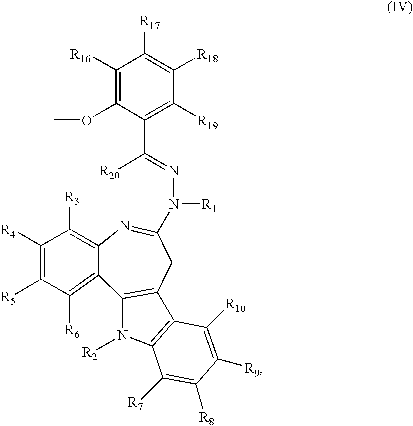
wherein
R¹ and R² are defined as above,
R³-R¹⁰ and R¹⁶-R²⁰ are defined as above, und
Mₐ is Ga, Fe, Ru or La,
Y is a physiologically compatible anion,
i is 2 or 3,
n is 1 or 2,
x is 0, 1, 2 or 3,
z is 1, 2 or 3,
nx+z≦i,
and physiologically compatible addition salts thereof.

4. Complex of the general formula (III′),
[MₐL₂]^{j+}j/p Y^{p-} (III′),
wherein L is a group of the general formula (IV), 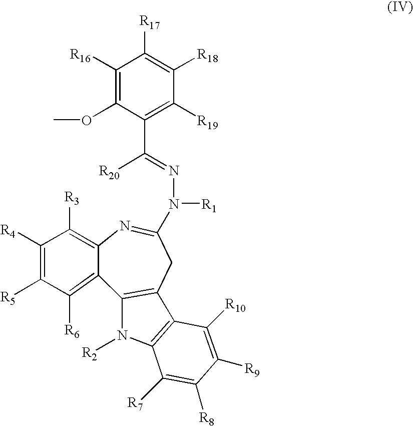
wherein
R¹ and R² are defined as in claim 1,
R³-R¹⁰ and R¹⁶-R²⁰ are defined as in claim 1,
Mₐ is Ga, Fe, La or Ru, and
j is 0, 1 or 2,
p is 1 or 2,
Y is a physiologically compatible anion,
and physiologically compatible addition salts thereof.

5. The complex according to claim 3, wherein Y is selected from the group consisting of halogen, pseudo-halogen, nitrate, carboxylate, sulphate and R″COO, wherein R″ is hydrogen, C₁-C₆-alkyl, C₂-C₆-alkenyl, C₂-C₆-alkynyl, C₃-C₆-cycloalkyl, C₃-C₆-cycloalkenyl, C₆-C₁₄-aryl, or a heterocycle.

6. The complex according to claim 5, wherein Y is chlorine.

7. The compound or complex according to claim 1, wherein R¹-R⁶ and R¹¹-R²⁰ in the general formulae (I), (II) or (IV) are hydrogen and R⁷-R¹⁰ are selected independently of one another from the group consisting of hydrogen, amino, nitro, cyano, formyl, carboxyl, SO₃H, SO₃ M_{b}, where M_{b} is a physiologically compatible cation, hydroxy, halogen, substituted or unsubstituted cycloalkyl, cycloalkenyl, aryl, substituted or unsubstituted, linear or branched alkyl, alkenyl, alkynyl, alkoxy, alkylmercapto and dialkylamino, wherein at least one substituent R⁷-R¹⁰ is not equal to hydrogen.

8. The compound or complex according to claim 7, wherein R⁷-R¹⁰ are selected independently of one another from the group consisting of hydrogen, C₁-C₆-alkyl, C₂-C₆-alkenyl, C₂-C₆-alkynyl, C₃-C₆-cycloalkyl, C₃-C₆-cycloalkenyl, C₆-C₁₄-aryl, amino, nitro, cyano, formyl, carboxyl, SO₃H, SO₃ M_{b}, wherein M_{b} is a physiologically compatible cation, hydroxy, halogen, C₁-C₄-halogenalkyl, C₁-C₄-hydroxyalkyl, C₁-C₄-alkylnitrile, C₁-C₄-alkoxy, C₁-C₄-alkoxy-C₁-C₄-alkylene, C₁-C₄-alkylmercapto, C₁-C₄-alkylmercapto-C₁-C₄-alkylene, C₁-C₄-alkoxycarbonyl, C₁-C₄-alkoxycarbonyl-C₁-C₄-alkylene, di-C₁-C₄-alkylamino, di-C₁-C₄-alkylamino-C₁-C₄-alkylene, di-C₁-C₄-alkylaminocarbonyl and di-C₁-C₄-alkylaminocarbonyl-C₁-C₄-alkylene.

9. The compound or complex according to claim 8, wherein R¹-R⁸ and R¹⁰—R²⁰ in the general formulae (I), (II) or (IV) are hydrogen and R⁹ is nitro, cyano, halogen or trifluoromethyl.

10. The compound or complex according to claim 9, wherein R⁹ is nitro or halogen.

11. The compound or complex according to claim 10, wherein the halogen is bromine.

12. The compound according to claim 1, wherein the compound of the general formula (I) is:

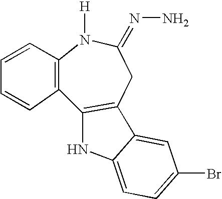

13. The compound according to claim 1, wherein the compound of the general formula (I) is:

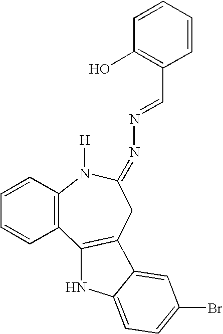

14. The compound according to claim 1, wherein the compound of the general formula (II) is:

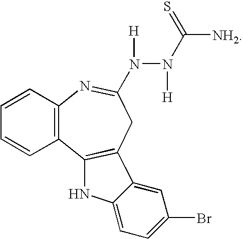

15. The compound according to claim 1, wherein the compound of the general formula (II) is:

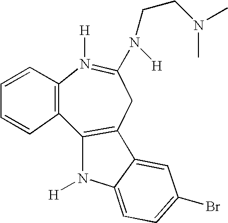

16. The compound according to claim 1, wherein the compound of the general formula (II) is:

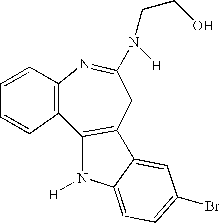

17. The compound of the general formula (VI) 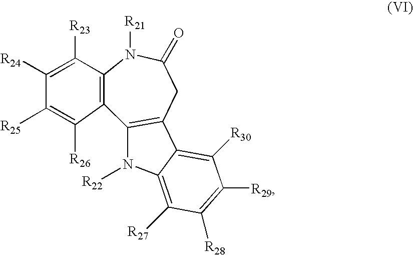
wherein
R²¹ and R²² are selected independently of one another from the group consisting of hydrogen, halogen, hydroxyl and substituted or unsubstituted cycloalkyl, cycloalkenyl, aryl, substituted or unsubstituted, linear or branched alkyl, alkenyl and alkynyl;
R²³ to R³⁰ are selected independently of one another from the group consisting of hydrogen, amino, nitro, cyano, formyl, carboxyl, hydroxy, halogen, SO₃H, SO₃ M_{b}, wherein M_{b} is a physiologically compatible cation, and substituted or unsubstituted cycloalkyl, cycloalkenyl, aryl, substituted or unsubstituted, linear or branched alkyl, alkenyl, alkynyl, alkoxy, alkylmercapto and dialkylamino, wherein at least one of the groups R²³ to R³⁰ is SO₃H or SO₃ M_{b}, wherein M_{b} is a physiologically compatible cation,
and physiologically compatible addition salts thereof.

18. Compound according to claim 17, wherein R²⁹ is SO₃H, or SO₃ M_{b}, wherein
M_{b} is a physiologically compatible cation.

19. Compound according to claim 17, wherein R²¹ to R²⁸ and R³⁰ are hydrogen.

20. Medicament containing a compound or a complex according to claim 1.

21. Use of a compound or complex according to claim 1 for the prophylaxis and/or treatment of tumor diseases.

22. Method for the manufacture of a complex of the general formula (Il)
[Mₐⁱ⁺Yₓⁿ⁻L_{z}]^{[i-(nx+z)]+}[i−(nx+z)]/n Yⁿ⁻ (III)
wherein a compound L of the general formula (IV), 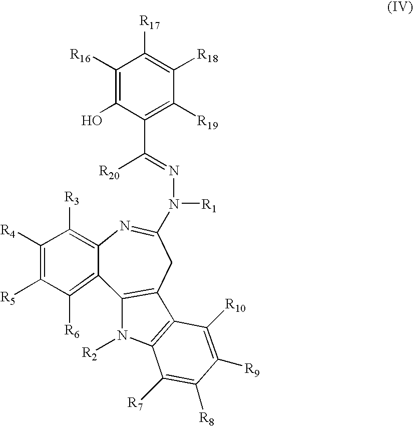
wherein
R¹-R¹⁰ and R¹⁶-R²⁰ are defined as in claim 1,
is reacted with a compound of the general formula (V),
MₐYₘ (V),
wherein
Mₐ is Ga, Fe, Ru or La,
Y is a physiologically compatible anion, and m is 1, 2 or 3, and
i is 2 or 3,
n is 1 or 2,
x is 0, 1, 2 or 3,
z is 1, 2 or 3.

23. Method for the manufacture of a complex of the general formula (III′)
[MₐL₂]^{j+}j/p Y^{p-} (III′),
wherein a compound L of the general formula (IV) 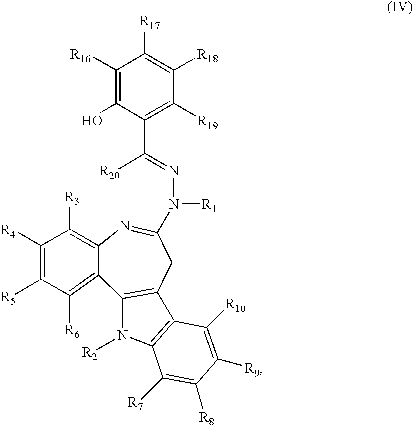
wherein
R¹ and R² are defined as in claim 1,
R³-R¹⁰ and R²⁰-R¹⁶ are defined as in claim 1,
is reacted with a compound of the general formula (V′),
MₐY_{q} (V′),
wherein
Mₐ is Ga, Fe, Ru or La,
Y is a physiologically compatible anion, and
q is 1, 2 or 3,
j is 0, 1 or 2
and p is 1 or 2.
